(19) United States
(12) Patent Application Publication
(10) Pub. No.: US 2026/0240779 A1
(43) Pub. Date: Aug. 20, 2026

(54) A STABLE TABLET-IN-TABLET COMPOSITION FOR NAUSEA VOMITING IN PREGNANCY

(71) Applicant: AKUMS DRUGS & PHARMACEUTICALS LIMITED, Delhi (IN)

(72) Inventors: Sanjeev Jain, Delhi (IN); Arun Chaudhary, Delhi (IN); Mukesh Dhiman, Delhi (IN)

(73) Assignee: AKUMS DRUGS & PHARMACEUTICALS LIMITED, Delhi (IN)

(21) Appl. No.: 19/160,479

(22) PCT Filed: Nov. 29, 2023
(86) PCT No.: PCT/IN2023/051112

§ 371 Date: Aug. 28, 2025

(30) Foreign Application Priority Data
Mar. 1, 2023 (IN) 202311013962

(51) Int. Cl. A61K 9/24; A61J 3/00; A61J 3/10; A61K 9/20; A61K 31/44; A61K 31/4402; A61P 1/08

(52) U.S. Cl. CPC A61K 9/209; A61J 3/005; A61J 3/10; A61K 9/2027; A61K 31/44; A61K 31/4402; A61P 1/08

(57) ABSTRACT

The present invention provides a stable pharmaceutical composition comprising fixed dose combination of doxylamine succinate and pyridoxine hydrochloride for effective treatment of nausea vomiting in pregnancy wherein the stable dosage form is a tablet-in-tablet formulation. The tablet-in-tablet dosage form of the present invention shows dual release having delayed release inner core table comprising doxylamine succinate and pyridoxine hydrochloride and an immediate release outer tablet compressed over inner tablet comprising doxylamine succinate and pyridoxine hydrochloride.

### FIELD OF INVENTION

The present invention relates to pharmaceutical composition comprising fixed dose combination of doxylamine and pyridoxine or pharmaceutically acceptable salts thereof. More particularly, the invention relates to the pharmaceutical composition in form of tablet-in-tablet composition and process of preparation thereof.

### BACKGROUND OF THE INVENTION

“Morning sickness” often known as nausea and vomiting of pregnancy, is a condition mostly occur during pregnancy and more than 80% of women experience nausea and/or vomiting. Non-pharmacological approaches such as use of ginger, aromatherapy, acupuncture are usually used to provide comport and treat the symptoms. However, most of the time non-pharmaceutical approaches are not sufficient enough to provide comfort and hence pharmaceutical approaches are required to be adopted for effective management.

DICLEGIS and BONJESTA are only FDA approved pharmacological treatment available in Market. DICLEGIS is a delayed release tablet comprising fixed dose combination of 10 mg doxylamine succinate and 10 mg pyridoxine hydrochloride used for treatment of nausea and vomiting of pregnancy in women who do not respond to conservative management wherein BONJESTA is an extended release tablet comprising fixed dose combination of 20 mg doxylamine succinate and 20 mg pyridoxine hydrochloride. BONJESTA is a film coated, multilayer tablet composition which comprises an enteric-coated core containing 10 mg doxylamine succinate and 10 mg pyridoxine hydrochloride, and an immediate release coating of 10 mg doxylamine succinate and 10 mg pyridoxine hydrochloride.

Delayed release formulation of doxylamine succinate and pyridoxine hydrochloride has already been disclosed in prior arts. Following are the few examples of such prior arts:
WO2022254277A1 discloses an extended release single layer matrix tablet composition of doxylamine succinate and pyridoxine hydrochloride comprising immediate release blend and delayed release granules.
US2016058709A1 discloses multi-layer composition comprising a core having combination of doxylamine and pyridoxine wherein the core is coated with enteric coating and the enteric coating layer is further coated with first active-ingredient coating wherein said first active ingredient is pyridoxine. The first active ingredient coating is further coated with intermediate coating layer which is further coated with second active ingredient coating layer wherein second active ingredient is doxylamine.

Almost all of the existing prior art teaches the preparation composition by multilayer or single layer tablet technologies. Even though progress has been made in developing multi-layered matrix tablet formulations, several issues are still being faced by formulator which includes layer separation, insufficient hardness, inaccurate individual layer weight control, and cross-contamination between the layers. The Separation of each individual layer in a multi-layered tablet is a common problem and significantly impacts the quality and efficacy of the product. It can occur during the compression and/or the dissolution process. The main cause of the separation can be insufficient bonding between the adjacent layers during the tablet compression. However, this issue can be relieved by assessing the force required to separate the layers of a multi-layered tablet. This might help to identify why each layer separates and to consequently take corrective action effectively. Manufacture of such a formulation and its scale-up are also challenging and a thorough understanding of tablet press design, process parameters and formulation components is required.

Thus there is still needs in the art for alternative formulations that overcomes the problems associated with multi-layered tablets and allow convenient administration of both the drugs as a single tablet. To overcome the above issues, the inventors have provided herewith a novel stable tablet-in-tablet pharmaceutical composition comprising fixed dose combination of doxylamine and pyridoxine which is simple, cost effective and overcome the complexity existed in the marketed dosage form to the manufacturer. The tablet-in-tablet formulation of the present invention provide an enhanced stability and provides less variation in serum concentration of the active agents due to uniformity content as compared to existing multi-layer technology.

### Objective

An objective of the present invention is to provide a pharmaceutical composition comprising two separate individual solid units, wherein the two individual units are a solid inner core and an outer solid shell and wherein the outer solid shell is compressed over the solid inner core to form a single unit solid pharmaceutical tablet.

The objective of the present invention is to formulate a stable pharmaceutical formulation comprising fixed dose combination of doxylamine and pyridoxine or its pharmaceutical acceptable salts thereof in a single dosage form and process of preparation thereof.

Another objective of the present invention is to provide a dosage form to overcome the complexity associated with multi-layer technology and able to develop a simple and cost effective technology in a single dosage form more preferably in form of solid dosage form more preferably tablet, capsule, pills, granules or sachets.

Yet another objective of the present invention is to provide oral dosage form comprising Doxylamine Succinate and Pyridoxine Hydrochloride by formulating a tablet-in-tablet dosage form. The tablet in tablet formulation of the present invention provides dual release i.e. immediate release followed by delayed release of the active agents wherein the formulation provides effective treatment of Nausea and vomiting of pregnancy (NVP) or “morning sickness”.

Another objective of the present invention is to provide tablet-in-tablet dosage form wherein the formulation comprising inner core tablet having fixed dose combination of doxylamine succinate and pyridoxine hydrochloride present in range 10 to 20 wt. % based on total weight of inner core tablet and an outer shell comprising doxylamine succinate and pyridoxine hydrochloride present in range 1 to 10 wt. % based on total weight of outer shell wherein outer shell is compressed over inner core tablet wherein outer shell releases the active agents in immediate form and inner core tablet releases the active agents in delayed form.

Summary: [0014] The present invention discloses an oral dosage form comprising fixed dose combination of two or more active agents in a single dosage form providing different release profiles. The composition of the present invention provides dual release of the two active agents in form of tablet-in-tablet composition. The present invention provides a tablet-in-tablet dosage form comprising an inner core tablet having fixed dose combination of Doxylamine succinate and Pyridoxine hydrochloride and outer shell comprising fixed dose combination of Doxylamine Succinate wherein inner core tablet releases in delayed manner and outer shell releases the active agent in immediate form. The tablet-in-tablet dosage form of present invention shows less serum concentration variation of the active agents and is more stable than existing multi-layer technology.

### BRIEF DESCRIPTION OF DRAWINGS

FIG. 1 represents the photography of tablet-in-tablet dosage form of present invention prepared from the illustrated examples in comparison with multi-layer technology of marketed product (Bonjesta).

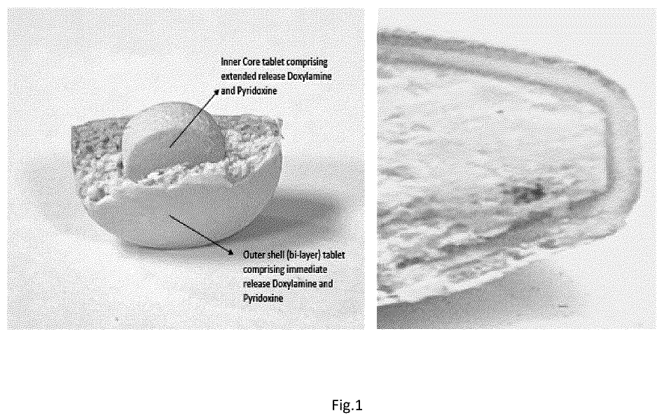

FIG. 2 represents the Linear Plot of Mean Plasma Concentrations of Doxylamine vs. Time for Test Product (T) and Reference Product (R) (N=34).

FIG. 3 represents the Ln-Linear Plot of Mean Plasma Concentrations of Doxylamine vs. Time for Test Product (T) and Reference Product (R) (N=34).

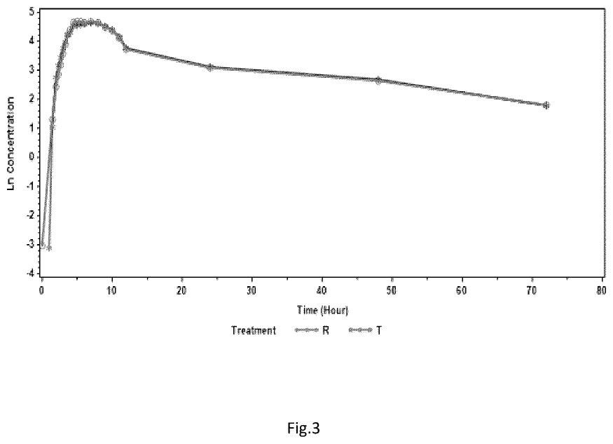

FIG. 4 represents the Linear Plot of Mean Plasma Concentrations of Pyridoxine vs. Time for Test Product (T) and Reference Product (R) (N=30).

FIG. 5 represents the Ln-Linear Plot of Mean Plasma Concentrations of Pyridoxine vs. Time for Test Product (T) and Reference Product (R) (N=30).

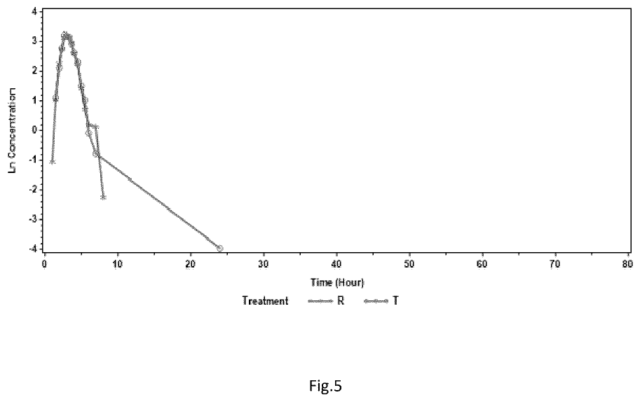

### DETAILED DESCRIPTION

The term “pharmaceutical composition” as used herein refers to delivery system in which active agents are delivered to the patients. This could be in the form of tablet, capsule, injection, liquid etc.

The term “pharmaceutically acceptable excipient” as used herein refers to inert substances other than active ingredients which are used in the preparation of pharmaceutical products.

The term “inner solid core” or “inner tablet” as used herein refers to single solid pharmaceutical dosage form containing the active medicaments and pharmaceutically acceptable excipients, and is prepared by compression of dry powder mix or granules prepared by wet granulation or dry granulation wherein the granules maybe optionally coated. Suitably, the “inner core” is a compact tablet.

The term “single unit solid pharmaceutical tablet” as used herein refers to single solid pharmaceutical dosage form containing the active medicament and pharmaceutically acceptable excipients

The term “outer shell” or “outer tablet” as used herein refers to single solid pharmaceutical dosage form containing the active medicament and pharmaceutically acceptable excipients mechanically compressed over the inner solid core or inner tablet,

The term “tablet-in-tablet” refers to formulation comprising an inner core tablet covered by an outer shell wherein outer shell compressed over inner core tablet.

The term “enteric coating” or “enteric coated” as used herein refers to gastro-resistant coating which prevents the dissolution or disintegration of oral formulation in gastric pH. Enteric coating is a basically a polymer coating which is applied to oral formulation to safely deliver drug to intestinal tract.

The present invention provides a stable pharmaceutical composition comprising fixed dose combination of two or more active agents wherein the combination of active agents presents in a single dosage form.

In one embodiment the present invention provides an oral dosage form comprising fixed dose combination of two active agents wherein oral dosage form is a solid dosage form selected from group consisting of table, capsule, granules or pills. In most preferred embodiment the solid dosage form is a tablet-in-tablet dosage form.

In an another embodiment the two or more active agents are a combination of antihistamine and vitamin B6.

In an embodiment antihistamine are selected from group consisting of Azelastine, Brompheniramine, Chlorpheniramine, Clemastine, Cyproheptadine, Dexchlorpheniramine, Diphenhydramine, Doxylamine, Loratadine, Fexofenadine or pharmaceutical acceptable salts thereof. In most preferred embodiment the active agent is Doxylamine or pharmaceutically acceptable salts thereof wherein succinate salt is being preferred.

In another embodiment another active agent is selected form vitamin B6 also known as pyridoxine and pharmaceutically acceptable salts thereof wherein hydrochloride salt is being preferred. The present invention provides a fixed dose combination of Doxylamine succinate and pyridoxine hydrochloride for effective management of Nausea and vomiting (NVP) of pregnancy.

Doxylamine is a H1-receptor anatagonist which blocks histamine production and decreases stimulation of vomiting center wherein pyridoxine (Vitamin B6) improves mild to moderate nausea, thus fixed dose combination of doxylamine succinate and pyridoxine hydrochloride provides a synergistic anti-nausea effects in pregnant women. Thus the present invention provides a fixed dose combination of doxylamine succinate and pyridoxine hydrochloride in a single dosage form.

In most preferred embodiment the present invention provides a fixed dose combination of doxylamine succinate and pyridoxine hydrochloride in a tablet-in-tablet dosage form wherein the dosage form provides delayed release and immediate release of the active agents.

In another embodiment the present invention provides a stable tablet-in-tablet pharmaceutical formulation comprising:
a) an inner core tablet comprising fixed dose combination of doxylamine succinate and pyridoxine hydrochloride
b) Outer shell comprising fixed dose combination of doxylamine succinate and pyridoxine hydrochloride wherein outer shell is compressed over inner core tablet to form tablet-in-tablet dosage form.

In an another embodiment the present invention provides a tablet-in-tablet dosage form comprising an inner core tablet and outer shell which is explained in detailed individually:

### Inner Core Tablet:

In an embodiment an inner core tablet comprises fixed dose combination of doxylamine succinate and pyridoxine hydrochloride present in range 10 to 20 wt. % based on total weight of inner core tablet more preferably 15.5 wt. % Doxylamine succinate and 16.92 wt. % Pyridoxine hydrochloride based on total weight of inner core tablet.

In an another embodiment an inner core tablet further comprises one or more pharmaceutical acceptable excipients wherein pharmaceutical acceptable excipients are selected from binder, diluent, disintegrant, lubricant, and wetting agent.

In another embodiment the inner core tablet comprises binder selected from but not limited to microcrystalline cellulose, starch, pregelatinized starch, polyethylene glycol (PEG), sorbitol, celluloses for example hydroxypropyl methylcellulose (HPMC); hydroxy ethylcellulose, hydroxypropyl cellulose, methylcellulose, and ethylcellulose, Polyvinylpyrrolidone (also known as polyvidone or povidone), or combination thereof. In most preferred embodiment the binder is Polyvinylpyrrolidone (PVPPK-30) present in an amount 0.5-5 wt. % based on total weight of inner core tablet more preferably 0.75 wt. % based on total weight of inner core tablet

In an embodiment the inner core tablet further comprises diluent selected from but not limited to selected from lactose e.g., directly compressible lactose, lactose monohydrate, lactose anhydrous, and spray dried lactose; microcrystalline cellulose sugar alcohols, e.g., sorbitol, erythritol, xylitol, and mannitol, dibasic calcium or potassium phosphate, starch, microcrystalline cellulose or combination thereof. In most preferred embodiment the diluents are microcrystalline cellulose, starch and Dibasic calcium phosphate present in an amount 5-40 wt. % based on total weight of inner core tablet.

In an embodiment the inner core tablet further comprises disintegrant selected from but not limited to croscarmellose sodium, crospovidone, starch, potato starch, pregelatinized starch, corn starch, sodium starch glycolate, microcrystalline cellulose, low substituted hydroxypropyl cellulose or combinations thereof. In most preferred embodiment disintergrants are croscarmellose sodium, crosspovidone and sodium starch glycollate present in an amount 0.5-5 wt. % based on total weight of inner core tablet.

In an another embodiment inner core tablet further comprises glidants selected from but not limited to calcium phosphate tribasic, magnesium silicate, colloidal silicon dioxide, talc or combination thereof. In most preferred embodiment the glidant is colloidal silicon dioxide present in an amount 0.5-3 wt. % based on total weight of inner core tablet more preferably 1.89 wt. % based on total weight of inner core tablet

In another embodiment the inner core tablet further comprises wetting agent selected from but not limited to polysorbate 80 (Tween 80), polysorbate 60, poloxamers polysorbate, 40, polysorbate 20, cremophors, tyloxapols, poloxamers, benzalkonium chloride, benzethonium chloride, cetyl alcohol, carbomer, cholesterol cocamidopropyl betaine, glyceryl monostearate, lanolin alcohols, sodium lauryl sulfate, nonoxynol 9, octoxynol 40, polyoxyl 35 castor oil, polyoxyl 40 hydrogenated castor oil, polyoxyl 40 steate, sorbitan monolaurate, sorbitan monooleate, or a combination thereof. In most preferred embodiment wetting agent is sodium lauryl sulfate present in an amount 0.05 to 1 wt. % based on total weight of inner core tablet more preferably 0.90 wt. % based on total weight of inner core tablet.

In an another embodiment the inner core tablet comprises lubricant selected from group consisting but not limited to calcium stearate, glyceryl monostearate, glyceryl palmitostearate, magnesium stearate, sodium lauryl sulfate, sodium stearyl fumarate, zinc stearate, stearic acid, hydrogenated vegetable oil, polyethylene glycol, talcum or sodium benzoate or combinations thereof. In most preferred embodiment the lubricant are magnesium stearate and talcum present in amount 0.5-3 wt. % based on total weight of inner core tablet more preferably 1.13 wt. % based on total weight of inner core tablet.

In an embodiment the inner core tablet is coated with different functional coatings or films. Examples of such coatings or films include, but are not limited to, controlled release, delayed release, modified release, seal coatings, pH dependent, pH independent coatings, and any combinations thereof.

In an another embodiment the inner core tablet is coated with one or more seal coatings which is further coated with enteric coating.

In most preferred embodiment the inner core tablet is further coated with on more seal coating selected from Instacoat sol and opadry clear.

In an another embodiment the enteric polymer coating of the solid inner core comprises a enteric polymer selected from but not limited to hydroxypropyl methylcellulose phtalate (HMPCP), polyvinyl acetate phtalate (PVAP), hydroxypropylmethylcellulose acetate succinate (HPMCAS), alginate, carbomer, carboxymethylcellulose, methacrylic acid copolymer (Eudragit L, Eudragit S, Acrycoat), shellac, cellulose acetate phthalate (CAP), starch glycolate, polacrylin, methyl cellulose acetate phtalate, hydroxypropyulcellulose acetate phthalate, cellulose acetate terephtahalate, cellulose acetate isophthalate and cellulose acetate trimellitate and wherein the enteric coated layer further comprises pharmaceutically acceptable excipient selected from additional polymer, pH modifier, lubricant, opacifier, surfactant, colorant and solvent.

In most preferred embodiment the enteric coating of inner core tablet provides delayed release of the active agents wherein the delayed release material is a polymer selected from methacrylic acid copolymer most preferably Eudragit L present in about 5-10 wt. % more preferably 5.69 wt. % based on total weight of inner core tablet.

In an embodiment the present invention provides tablet-in tablet dosage form comprising an inner core having:
i. From about 15.5 wt. % Doxylamine succinate and 16.92 wt. % Pyridoxine hydrochloride based on total weight of inner core tablet;
ii. From about 0.75 wt. % Polyvinylpyrrolidone (PVPPK-30) based on total weight of inner core tablet as binder;
iii. From about 5-40 wt. % microcrystalline cellulose, starch and Dibasic calcium phosphate based on total weight of inner core tablet as diluents;
iv. From about 0.5-5 wt. % of croscarmellose sodium, crosspovidone and sodium starch glycollate based on total weight of inner core tablet as disintegrant;
v. From about 1.89 wt. % of colloidal silicon dioxide based on total weight of inner core tablet as glidant;
vi. From about 0.90 wt. % of Sodium Lauryl sulfate based on total weight of inner core tablet as wetting agent;
vii. From about 1.13 wt. % of Magnesium stearate based on total weight of inner core tablet as lubricant;
viii. From about 5.69 wt. % of Eudragit L as enteric coated polymer which provides delayed release of active agents.

In an embodiment the present invention provides a process of preparation of inner core tablet which comprises following process steps:
a) Dry mixing of active agents with diluents and disintegrant.
b) Wet granulating the premix with binder solution to form granules
c) Drying the wet granules and lubricating the dried granules
d) Compressing the lubricating granule into a tablet
e) Coating the tablet with one more seal coating
f) Enteric coating the seal granules with delayed release polymer

### Outer Shell/Tablet:

The present invention provides an outer shell surrounding inner core tablet wherein out shell is compressed with inner core tablet to form tablet-in-tablet formulation.

In an embodiment the outer shell of the present invention comprises fixed dose combination of doxylamine succinate and pyridoxine hydrochloride present in range 1 to 10 wt. % based on total weight of outer shell and one or more pharmaceutically acceptable excipients.

In an another embodiment the pharmaceutical acceptable excipients are selected from diluent, disintegrant, binder, lubricant, glidant and colorant.

In an embodiment the outer shell of the present invention comprise one or more diluents selected from but not limited to lactose e.g., directly compressible lactose, lactose monohydrate, lactose anhydrous, and spray dried lactose; microcrystalline cellulose sugar alcohols, e.g., sorbitol, erythritol, xylitol, and mannitol, dibasic calcium or potassium phosphate, starch, microcrystalline cellulose or combination thereof. In most preferred embodiment the diluents are microcrystalline cellulose, starch and Dibasic calcium phosphate present in an amount 10-50 wt. % based on total weight of outer shell.

In an another embodiment the outer shell of the present invention comprises one or more disintegrant selected from but not limited to croscarmellose sodium, crospovidone, starch, potato starch, pregelatinized starch, corn starch, sodium starch glycolate, microcrystalline cellulose, low substituted hydroxypropyl cellulose or combinations thereof. In most preferred embodiment disintergrant is croscarmellose sodium, present in an amount 0.5-5 wt. % based on total weight of outer shell.

In another embodiment the outer shell further comprises binder selected from but not limited to microcrystalline cellulose, starch, pregelatinized starch, polyethylene glycol (PEG), sorbitol, celluloses for example hydroxypropyl methylcellulose (HPMC); hydroxy ethylcellulose, hydroxypropyl cellulose, methylcellulose, and ethylcellulose, Polyvinylpyrrolidone (also known as polyvidone or povidone), or combination thereof. In most preferred embodiment the binder is Polyvinylpyrrolidone (PVPPK-30) present in an amount 0.5-5 wt. % based on total weight of outer shell.

In an another embodiment the outer shell further comprises lubricant selected from group consisting but not limited to calcium stearate, glyceryl monostearate, glyceryl palmitostearate, magnesium stearate, sodium lauryl sulfate, sodium stearyl fumarate, zinc stearate, stearic acid, hydrogenated vegetable oil, polyethylene glycol, talcum or sodium benzoate or combinations thereof. In most preferred embodiment the lubricant is magnesium stearate and talcum present in amount 0.05-2 wt. % based on total weight of outer shell.

In an another embodiment outer shell further comprises glidants selected from but not limited to calcium phosphate tribasic, magnesium silicate, colloidal silicon dioxide, talc or combination thereof. In most preferred embodiment the glidant is colloidal silicon dioxide present in an amount 0.5-3 wt. % based on total weight of outer shell.

In an embodiment the present invention provides tablet-in tablet dosage form comprising an outer shell having:
i. From about 1-10 wt. % Doxylamine succinate and Pyridoxine hydrochloride based on total weight of outer shell and;
ii. From about 10-50 wt. % microcrystalline cellulose, starch and Dibasic calcium phosphate based on total weight of outer shell as diluents and;
iii. From about 0.5-5 wt. % croscarmellose sodium based on total weight of outer shell as disintegrate and;
iv. From about 0.5-5 wt. % Polyvinylpyrrolidone (PVPPK-30) based on total weight of outer shell as binder and;
v. From about 0.05-2 wt. % magnesium stearate and talcum based on total weight of outer shell as lubricant and;
vi. From about 0.5-3 wt. % colloidal silicon dioxide based on total weight of outer shell as glidant

In an another embodiment the outer shell may be further coated with seal coating and optionally comprising colorant wherein seal coating comprises plasticizer, film former and solvent.

In an another embodiment the outer shell of the present invention may be present in multi-layer for example bi-layer or tri-layer.

In a preferred embodiment the outer shell is bi-layered and is composed of an upper layer and a lower layer wherein the amount of doxylamine succinate and pyridoxine hydrochloride is split between the upper layer and the lower layer is in the ratio of 1:1.

In an another embodiment the outer shell of the present invention provides immediate release of the active agents.

In an another embodiment the present invention provides a method of producing outer shell which comprises following process steps:
a) Dry mixing diluent and disintegrants with active agents
b) Wet granulating the dry mix with binder solution to form wet granules
c) Drying the wet granules and lubricated the dried granules with the lubricants
d) Compressing the dried granules with inner core tablet to form tablet-in-tablet formulation
e) coating the outer shell of tablet-in-tablet with seal coating

In an embodiment the present invention provides a tablet-in-tablet formulation comprises:
a. A delayed release inner core tablet comprising 10 mg Doxylamine succinate and 10 mg Pyridoxine hydrochloride
b. An immediate release outer shell comprising 10 mg Doxylamine succinate and 10 mg Pyridoxine hydrochloride.

Wherein an outer shell compressed with inner core tablet to form tablet-in-tablet dosage form and is used for the treatment of nausea and vomiting in pregnancy in women who do not respond to conservative management.

In an another embodiment of the present study the tablet-in-tablet dosage form is better than existing multi-layer technology as it shows less serum concentration variation of active agents with improved stability.

While the invention has been described with respect to specific composition which include presently preferred modes of carrying out the invention, those skilled in the art will appreciate that there are numerous variations and permutations of the above described embodiments that fall within the spirit and scope of the invention. It should be understood that the invention is not limited in its application to the details of construction and arrangements of the components set forth herein.

### EXAMPLES

The present invention is described in detail with reference to the examples given below. The examples are provided just to illustrate the invention and therefore, should not be construed to limit the scope of the invention.

### Example-1: Formula for 10 mg Doxylamine Succinate and 10 mg Pyridoxine Hydrochloride Inner Core Tablet

TABLE 1
Composition details of inner core tablet

| | | | |
|---|---|---|---|
| S. No. | Ingredients | % age | Function |

INNER CORE TABLET
DRY MIX

| | | | |
|---|---|---|---|
| 1 | Doxylamine Succinate | 15.538 | Active |
| 2 | Pyridoxine Hydrochloride | 16.92 | Active |
| 3 | Microcrystalline Cellulose | 30.80 | Diluent |
| 4 | Starch (Maize) | 15.152 | Diluent |
| 5 | Dibasic Calcium Phosphate | 6.0606 | Diluent |
| | (Dihydrate) | | |
| 6 | Croscarmellose Sodium | 1.8182 | Disintegrant |

BINDER

| | | | |
|---|---|---|---|
| 7. | Polyvinyl pyrrolidone K-30 | 0.7576 | Binder |
| | (PVPK-30) | | |
| 8. | Purified water | — | Solvent |

LUBRICATION

| | | | |
|---|---|---|---|
| 9 | Croscarmellose Sodium | 2.4242 | Disintegrant |
| 10 | Talcum | 1.1364 | Lubricant |
| 11 | Colloidal Silicon Dioxide | 1.8939 | Glidant |
| 12. | Crosspovidone INF-10 | 3.6364 | Disintegrant |
| 13. | Sodium Starch Glycollate | 3.3333 | Disintegrant |
| | (Primojel) | | |
| 14. | Sodium Lauryl Sulfate | 0.9091 | Wetting Agent |
| 15. | Magnesium Stearate | 1.1364 | Lubricant |

COATING
SEAL COATING 1^{st}

| | | | |
|---|---|---|---|
| 10. | Instacoat sol IC-S-1643 | Film Former | — |
| 11. | Iso Propyl Alcohol | Solvent | — |
| 12. | Methylene Chloride | Solvent | — |

SEAL COATING 2^{nd}

| | | | |
|---|---|---|---|
| 13 | Opadry Clear 1R 7006 | Film Former | — |
| | (Colorcon) | | |
| 14 | Iso Propyl Alcohol | Solvent | — |
| 15 | Methylene Chloride | Solvent | — |

ENTERIC COATING

| | | | |
|---|---|---|---|
| 16. | Eudragit L 100-55 | Delayed | — |
| | | Release | |
| | | material | |
| 17. | Triacetin | Polymer | — |
| 18. | Sodium Hydroxide (Pellets) | pH Modifier | — |
| 19. | Talcum | Lubricant | — |
| 20. | Titanium Dioxide | Opacifier | — |
| 21. | Polysorbate 80 (Tween 80) | Surfactant | — |
| 22. | Iron Oxide (Ferric Oxide ) Red | Colour | — |
| 23. | Purified Water | Solvent | — |

### Example-2: Formula for 10 mg Doxylamine Succinate and 10 mg Pyridoxine Hydrochloride Outer Tablet

TABLE 2
Composition details of outer tablet

| | | | |
|---|---|---|---|
| S. No. | Ingredients | % age | Function |

OUTER SHELL/TABLET
DRY MIX

| | | | |
|---|---|---|---|
| 1 | Doxylamine Succinate | 1.86 | Active |
| 2 | Pyridoxine Hydrochloride | 2.02 | Active |
| 3 | Microcrystalline Cellulose | 46.78 | Diluent |
| 4 | Starch (Maize) | 16.36 | Diluent |
| 5 | Dibasic Calcium Phosphate | 24.56 | Diluent |
| | (Dihydrate) | | |
| 6 | Croscarmellose Sodium | 0.70 | Disintegrant |

BINDER

| | | | |
|---|---|---|---|
| 7. | Polyvinyl pyrrolidone K-30 | 3.72 | Binder |
| | (PVPK-30) | | |
| 8. | Purified water | — | Solvent |

LUBRICATION

| | | | |
|---|---|---|---|
| 9 | Croscarmellose Sodium | 2.80 | Disintegrant |
| 10 | Talcum | 0.85 | Lubricant |
| 11 | Colloidal silicon dioxide | 0.96 | Glident |
| 12. | Magnesium Stearate | 0.40 | Lubricant |

COATING (SEAL COATING)

| | | | |
|---|---|---|---|
| 13. | P.E.G.-6000 IP | Plasticizer | — |
| 14. | H.P.M.C E-15 | Film Former | — |
| 15. | Iso Propyl Alcohol (IPA) | Solvent | — |
| 16. | Methylene Chloride | Solvent | — |
| 17. | Ready Mix Coat. Mat. | Polymer | — |
| | (Opadry II 85F190000) | | |

### Example 3—a Process of Preparation of 20 mg Doxylamine Succinate and 20 mg Pyridoxine Hydrochloride

a) Preparation of Inner Core Tablet:
   i. Doxylamine succinate and Pyridoxine hydrochloride are dry mixed with microcrystalline cellulose, starch (Maize), dibasic calcium phosphate and croscarmellose sodium.
   ii. The premix obtained after sifting and dry mixing was wet granulated with binder solution (polyvinyl pyrrolidone K-30 with water).
   iii. The wet mass after drying was lubricated with croscarmellose sodium, talcum, crospovidone, sodium starch glycolate, colloidal silicon dioxide and magnesium sterate
   iv. The lubricated granules are compressed to form uncoated inner core tablet
   v. The coated inner core tablet was further coated with one or more seal coating,
   vi. The seal coated was further enteric coated with delayed release polymer to obtain delayed inner core tablet.
a. Preparation of Outer Shell/tablet:
   i. Doxylamine succinate and Pyridoxine hydrochloride are dry mixed with microcrystalline cellulose, starch (Maize), dibasic calcium phosphate and croscarmellose sodium.
   ii. The premix obtained after sifting and dry mixing was wet granulated with binder solution (polyvinyl pyrrolidone K-30 with water).
   iii. The wet mass after drying was lubricated with croscarmellose sodium, talcum, colloidal silicon dioxide and magnesium sterate.
   iv. The lubricated granules are compressed along with inner tablet wherein lubricated outer shell granules are compressed with inner core tablet to form tablet-in-tablet.

### Example 4—Formula for 10 mg Doxylamine Succinate and 10 mg Pyridoxine Hydrochloride Outer Bi Layer Tablet

TABLE 3
Composition details of upper layer of Outer bi layer tablet

| | | | |
|---|---|---|---|
| S.NO | Ingredients | % age | Function |

Doxylamine succinate & Pyridoxine Hydrochloride(Part-1) (Upper Layer)
Dry mixing

| | | | |
|---|---|---|---|
| 1 | Doxylamine succinate | 2.564 | API |
| 2 | Pyridoxine Hydrochloride | 2.792 | API |
| 3 | Microcrystalline Cellulose | 47.900 | Diluent |
| 4 | Dibasic Calcium Phosphate | 19.000 | Diluent |
| | (Dihydrate) | | |
| 5 | Starch (Maize) | 20.000 | Diluent |
| 6 | Croscarmellose Sodium | 1.000 | Disintegrant |

Binder Preparation

| | | | |
|---|---|---|---|
| 7 | Polyvinyl pyrrolidone (PVPK-30) | 3.250 | Binder |
| 8 | Purified water | — | Solvent |

Lubrication

| | | | |
|---|---|---|---|
| 9 | Croscarmellose Sodium | 2.750 | Disintegrant |
| 10 | Talcum | 0.850 | Lubricant |
| 11 | Colloidal Silicon Dioxide | 0.900 | Glidant |
| 12 | Magnesium Stearate | 1.000 | Lubricant |

TABLE 4
Composition details of lower layer of Outer bi layer tablet

| | | | |
|---|---|---|---|
| S.NO | Ingredients | % age | Function |

Doxylamine succinate & Pyridoxine Hydrochloride(Part-1) (Lower Layer)
Dry mixing

| | | | |
|---|---|---|---|
| 1 | Doxylamine succinate | 1.465 | API |
| 2 | Pyridoxine Hydrochloride | 1.595 | API |
| 3 | Microcrystalline Cellulose | 46.070 | Diluent |
| 4 | Dibasic Calcium Phosphate (Dihydrate) | 27.740 | Diluent |
| 5 | Starch (Maize) | 14.290 | Diluent |
| 6 | Croscarmellose Sodium | 0.571 | Disintegrant |
| 7 | Iron Oxide (Ferric Oxide) Red | 0.093 | Colour |

Binder Preparation

| | | | |
|---|---|---|---|
| 8 | Polyvinyl pyrrolidone (PVPK-30) | 4.000 | Binder |
| 9 | Purified Water | — | Solvent |

Lubrication

| | | | |
|---|---|---|---|
| 10 | Croscarmellose Sodium | 2.860 | Disintegrant |
| 11 | Talcum | 0.860 | Lubricant |
| 12 | Colloidal Silicon Dioxide | 1.000 | Glidant |
| 13 | Iron Oxide (Ferric Oxide) Red | 0.090 | Colour |
| 14 | Magnesium Stearate | 0.800 | Lubricant |

Coating

| | | | |
|---|---|---|---|
| 15 | P.E.G.-6000 IP | 0.840 | Plasticizer |
| 16 | H.P.M.C E-15 | 6.660 | Film Former |
| 17 | Iso Propyl Alcohol (IPA) | — | Solvent |
| 18 | Methylene Chloride 85 | — | Solvent |

After loss of Coating material wt.

| | | | |
|---|---|---|---|
| 19 | Ready Mix Coat. Mat. | 12.56 | Polymer |
| | (Opadry II 85F190000) | | |
| 20 | Purified Water | qs | solvent |

### Example 5—In-Vitro Drug Profile of 20 mg Doxylamine Succinate and 20 mg Pyridoxine Hydrochloride Tablet-In-Tablet Under Different Stability Conditions in Different Dissolution Media

a. Temperature 30° C.±2° C. & RH 75%±5%

TABLE 5
Percentage release of Doxylamine succinate at temperature
30° C. ± 2° C. & RH 75% ± 5% in acidic medium.

| | | Percentage of Doxylamine succinate Release | | | | | |
|---|---|---|---|---|---|---|---|
| Dis- | Time | | | | After | After | After |
| solution | (in | Initial | After 3 | After 6 | 9 | 12 | 18 |
| media | minutes) | Testing | months | months | months | months | months |
| Acidic | After 30 | 48.31 | 42.38 | 49.15 | 52.57 | 48.44 | 49.48 |
| media | minutes | | | | | | |
| | After | 51.78 | 46.41 | 50.13 | 52.83 | 48.25 | 50.71 |
| | 120 | | | | | | |
| | minutes | | | | | | |

TABLE 6
Percentage release of Pyridoxine hydrochloride at temperature
30° C. ± 2° C. & RH 75% ± 5% in acidic medium.

| | | Percentage of Pyridoxine hydrochloride Release | | | | | |
|---|---|---|---|---|---|---|---|
| Dis- | Time | | | | | After | After |
| solution | (in | Initial | After 3 | After 6 | After 9 | 12 | 18 |
| media | minutes) | Testing | months | months | months | months | months |
| Acidic | After 30 | 48.43 | 47.25 | 47.08 | 43.88 | 47.36 | 52.43 |
| media | minutes | | | | | | |
| | After | 53.28 | 49.62 | 47.06 | 44.60 | 48.37 | 55.42 |
| | 120 | | | | | | |
| | minutes | | | | | | |

TABLE 7
Percentage release of Doxylamine succinate at temperature
30° C. ± 2° C. & RH 75% ± 5% in buffer medium

| | | Percentage of Doxylamine succinate Release | | | | | |
|---|---|---|---|---|---|---|---|
| Dis- | | | | | | After | After |
| solution | Time (in | Initial | After 3 | After 6 | After 9 | 12 | 18 |
| media | minutes) | Testing | months | months | months | months | months |
| Buffer | After | 103.16 | 103.60 | 100.23 | 105.59 | 98.24 | 106.87 |
| Media | 180 | | | | | | |
| | min | | | | | | |
| | (120 | | | | | | |
| | min in | | | | | | |
| | acid and | | | | | | |
| | 60 min | | | | | | |
| | in | | | | | | |
| | buffer | | | | | | |

TABLE 8
Percentage release of Pyridoxine hydrochloride at temperature
30° C. ± 2° C. & RH 75% ± 5% in buffer medium

| | | Percentage of Pyridoxine HCI Release | | | | | |
|---|---|---|---|---|---|---|---|
| Dis- | Time | | | | | After | After |
| solution | (in | Initial | After 3 | After 6 | After 9 | 12 | 18 |
| media | minutes) | Testing | months | months | months | months | months |
| Buffer | After | 110.53 | 107.31 | 102.89 | 104.38 | 106.38 | 103.22 |
| Media | 180 | | | | | | |
| | min | | | | | | |
| | (120 | | | | | | |
| | min in | | | | | | |
| | acid | | | | | | |
| | and 60 | | | | | | |
| | min | | | | | | |
| | in buffer | | | | | | |

b. Temperature 25° C.±2° C. & RH 60%±5%

TABLE 9
Percentage release of Doxylamine Succinate at temperature
25° C. ± 2° C. & RH 60% ± 5% in acidic medium

| | | Percentage of Doxylamine succinate Release | | | | | |
|---|---|---|---|---|---|---|---|
| Dis- | | | | | | After | After |
| solution | Time (in | Initial | After 3 | After 6 | After 9 | 12 | 18 |
| media | minutes) | Testing | months | months | months | months | months |
| Acidic | After 30 | 48.31 | 44.65 | 49.17 | 52.50 | 50.75 | 49.72 |
| media | minutes | | | | | | |
| | After | 51.78 | 47.93 | 50.96 | 52.14 | 50.75 | 49.72 |
| | 120 | | | | | | |
| | minutes | | | | | | |

TABLE 10
Percentage release of Pyridoxine hydrochloride at temperature
25° C. ± 2° C. & RH 60% ± 5% in acidic medium

| | | Percentage of Pyridoxine hydrochloride Release | | | | | |
|---|---|---|---|---|---|---|---|
| Dis- | | | | | | After | After |
| solution | Time (in | Initial | After 3 | After 6 | After 9 | 12 | 18 |
| media | minutes) | Testing | months | months | months | months | months |
| Acidic | After 30 | 48.43 | 48.98 | 47.47 | 43.75 | 49.02 | 52.37 |
| media | minutes | | | | | | |
| | After | 53.28 | 50.40 | 46.19 | 44.69 | 49.31 | 51.37 |
| | 120 | | | | | | |
| | minutes | | | | | | |

TABLE 11
Percentage release of Doxylamine Succinate at temperature
25° C. ± 2° C. & RH 60% ± 5% in buffer medium

| | | Percentage of Doxylamine Succinate Release | | | | | |
|---|---|---|---|---|---|---|---|
| Dis- | | | | | | After | After |
| solution | Time (in | Initial | After 3 | After 6 | After 9 | 12 | 18 |
| media | minutes) | Testing | months | months | months | months | months |
| Buffer | After | 103.16 | 107.86 | 100.95 | 104.76 | 101.52 | 105.59 |
| Media | 180 | | | | | | |
| | min | | | | | | |
| | (120 | | | | | | |
| | min in | | | | | | |
| | acid | | | | | | |
| | and 60 | | | | | | |
| | min | | | | | | |
| | in buffer | | | | | | |

TABLE 12
Percentage release of Pyridoxine hydrochloride at temperature
25° C. ± 2° C. & RH 60% ± 5% in buffer medium

| | | Percentage of Pyridoxine Hcl Release | | | | | |
|---|---|---|---|---|---|---|---|
| Dis- | Time | | | | | After | After |
| solution | (in | Initial | After 3 | After 6 | After 9 | 12 | 18 |
| media | minutes) | Testing | months | months | months | months | months |
| Buffer | After | 110.53 | 105.87 | 103.11 | 103.85 | 97.29 | 100.28 |
| Media | 180 | | | | | | |
| | min | | | | | | |
| | (120 | | | | | | |
| | min in | | | | | | |
| | acid | | | | | | |
| | and 60 | | | | | | |
| | min | | | | | | |
| | in buffer | | | | | | |

### Example-6: Comparative Pharmacokinetic Study

The rate and extent of absorption of Doxylamine, pyridoxine from Doxylamine succinate and pyridoxine Hydrochloride tablet-in-tablet of the present invention referred as “test product” was compared marketed tablet of Bonjesta (Doxylamine succinate 20 mg and Pyridoxine Hydrochloride 20 mg Extended release tablets) referred as “reference product” of Duchesnay Bonjesta (Doxylamine succinate 20 mg and Pyridoxine Hydrochloride 20 mg Extended release tablets) of Duchesnay.

### Methodology

A randomized, open label, balanced, two treatments, two periods, two sequence, single dose, crossover design.

All study related procedures, duration, dates and timings, information on the study treatments and confidentiality of the subject's data were explained clearly to the subjects by clinical personnel during the informed consent procedure. Subjects who signed the consent form and showed their willingness to participate in the study were enrolled. Subjects who were eligible when assessed against the inclusion and exclusion criteria and who were found to be healthy on physical examination with laboratory investigation values within reference limits were considered for admission to the study. Subjects whose pre-study laboratory values were outside the reference range were also considered for participation provided these values were considered clinically non-significant by the investigator.

Treatments were allocated to subjects as per the randomization schedule generated using statistical techniques with SAS (SAS Institute Inc., USA) version 9.4. The pre-dose blood sample were collected at −3.00, −1.00, −0.50 within 02 minutes prior to dosing and 0.00 hour was collected within 10 minutes prior to dosing; the post-dose in-house samples were collected within 02 minutes of the scheduled sampling time. The blood sample at 48.00- and 72.00-hours post-dose were collected on ambulatory basis (i.e. on separate visit).

Statistical analysis was performed to assess bioequivalence between the pharmacokinetic parameters of test and reference formulations using SAS (SAS Institute Inc., USA) version 9.4.

### Number of Subjects

Thirty-six (36) healthy, adult, human female subjects were dosed in the study and 34 subjects completed the study.

### Method of Administration

After overnight fasting of 10.00 hours, one tablet either of test product (T) or reference product (R) was administered orally to the subjects in sitting posture with approximately 240 ml of water at ambient temperature in the morning, as per randomization schedule in each period, followed by a thorough mouth check to ensure that the drug had been swallowed. The subject was instructed not to chew or crush or divide the tablet but to swallow it whole.

### Duration of Study

The total duration of the clinical phase of the study was 33 days from the day of check-in of the first period till the end of the second period.

### Washout Period:

The administration of each product was followed by a washout period of 28 days (i.e., at least five elimination half-lives), thus minimizing chances of measurable levels of drug being present before dosing in the following period.

### Diet and Water:

Subjects were fasted for at least 10.00 hours prior to dosing. Standard meal was provided on the day of check-in and at approximately 4.00, 8.00 and 12.00 hours after dosing in each period. During housing; all meal plans were identical in each period.

Drinking water were prohibited for one hour before and one hour after dosing (except approximately 240 ml of water given during dosing). At other times, drinking water was provided ad libitum.

### Criteria for Evaluation:

Assessment of bioequivalence was done on the basis of the 90% confidence intervals of the geometric least squares treatment means for Ln-transformed Cₘₐₓ, AUC₀₋ₜ and AUC_{0-∞} for Doxylamine and Cₘₐₓ and AUC₀₋₇₂ for Pyridoxine.

The acceptance criteria for bioequivalence was that the entire confidence intervals for the geometric least squares of means of Ln-transformed Cₘₐₓ, AUC₀₋ₜ and AUC_{0-∞} for Doxylamine, and Cₘₐₓ, and AUC₀₋₇₂ for Pyridoxine should fall within 80.00-125.00%.

### Pharmacokinetic Parameters:

Employing the estimated plasma concentration-time profiles of Doxylamine and Pyridoxine the following pharmacokinetic parameters were calculated using SAS (SAS Institute Inc., U.S.A.) version.

### For Doxylamine:

Primary parameters: Cₘₐₓ, AUC₀₋ₜ and AUC_{0-∞}
Secondary parameters: tₘₐₓ, t_{1/2}, kₑₗ and extrapolated AUC

### For Pyridoxine:

Primary parameters: Cₘₐₓ, and AUC₀₋₇₂
Secondary parameters: Tₘₐₓ,

### Statistical Analysis:

Statistical analysis of the pharmacokinetic parameters was performed using SAS (SAS Institute Inc., U.S.A.) version 9.4.

Descriptive statistics were computed and reported for the pharmacokinetic parameters. The log-transformed pharmacokinetic parameters Cₘₐₓ, AUC₀₋ₜ and AUC_{0-∞} for Doxylamine, and Cₘₐₓ and AUC₀₋₇₂ for Pyridoxine were analysed using ANOVA. The Intra subject CV, Power, Ratio analysis and 90% confidence interval for the ratio of the geometric least squares mean were computed for log-transformed pharmacokinetic parameters Cₘₐₓ, AUC₀₋ₜ and AUC_{0-∞} for Doxylamine, and Cₘₐₓ and AUC₀₋₇₂ for Pyridoxine.

### Results

### A) Pharmacokinetic and Statistical Evaluation:

### Doxylamine

TABLE 13
Descriptive Statistics of Pharmacokinetic Parameters of Test
Product (T) and Reference Product (R) for Doxylamine
(N = 34)

| Form | Variable | Mean | SD |
|---|---|---|---|
| R | Cₘₐₓ (ng/mL) | 123.0569 | 21.8753 |
| | Tₘₐₓ (hr) | 5.9691 | 1.9132 |
| | AUC₀₋ₜ (ng · hr/mL) | 1838.8652 | 523.4598 |
| | AUC_{0-∞} (ng · hr/mL) | 2112.8353 | 688.5694 |
| | Kₑₗ (hr⁻¹) | 0.0339 | 0.0181 |
| | t_{1/2} (hr) | 23.1271 | 6.4081 |
| | AUC_%Extrap_obs | 11.7379 | 6.4349 |
| T | Cₘₐₓ (ng/mL) | 123.8944 | 24.3229 |
| | Tₘₐₓ (hr) | 5.2159 | 1.6353 |
| | AUC₀₋ₜ (ng·hr/mL) | 1827.7079 | 521.9363 |
| | AUC_{0-∞} (ng·hr/mL) | 2085.0987 | 613.6875 |
| | Kₑₗ (hr⁻¹) | 0.0324 | 0.0139 |
| | t_{1/2} (hr) | 24.6226 | 9.1708 |
| | AUC_%Extrap_obs | 11.8118 | 6.7920 |

From Table 13, the time to achieve maximum plasma concentration (Tmax) of Test product Doxylamine is about 5.2159 h whereas the reference Doxylamine is about 5.9691 h which showed that test product takes less time to achieve maximum concentration as compared to reference product. Also the single dose of test product 20 mg produces maximum plasma concentrations (Cmax) of about 123.8944 ng/ml whereas reference product produces maximum plasma concentrations (Cmax) of about 123.0569 ng/ml, thus the Cmax of test product is higher as compared to the reference product.

### Pyridoxine

TABLE 14
Descriptive Statistics of Pharmacokinetic Parameters of Test
Product (T) and Reference Product (R) for Pyridoxine
(N = 30)

| Form | Variable | Mean | SD |
|---|---|---|---|
| R | Cₘₐₓ (ng/mL) | 41.9760 | 12.1704 |
| | Tₘₐₓ (hr) | 54.7700 | 21.7098 |
| | AUC₀₋₇₂ | 3.0990 | 1.0154 |
| T | Cₘₐₓ (ng/mL) | 42.6889 | 9.7035 |
| | Tₘₐₓ (hr) | 54.7404 | 16.6237 |
| | AUC₀₋₇₂ (ng · hr/mL) | 3.0440 | 0.7815 |

From Table 14, the time to achieve maximum plasma concentration (Tmax) of Test product Doxylamine is about 54.77 h whereas the reference Doxylamine is about 54.74 h which showed that test product takes less time to achieve maximum concentration as compared to reference product. Also the single dose of test product 20 mg produces maximum plasma concentrations (Cmax) of about 42.6889 ng/ml whereas reference product produces maximum plasma concentrations (Cmax) of about 41.9760 ng/ml, thus the Cmax of test product is higher as compared to the reference product.

B) Mean Plasma Concentrations Vs. Time Curve

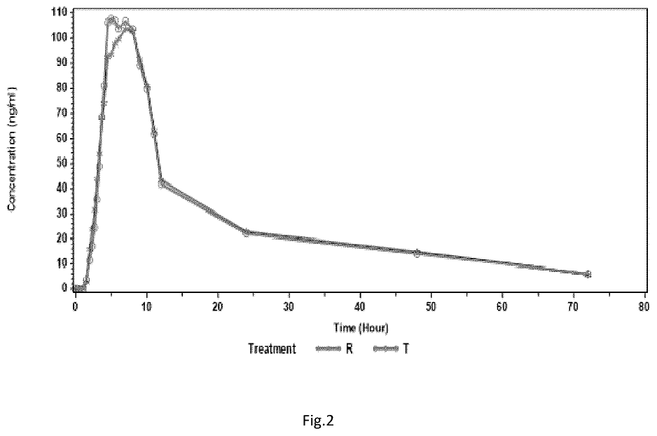

The FIG. 2 represent the liner plot of Mean Plasma Concentrations of Doxylamine vs. Time for Test Product (T) and Reference Product (R). From the graph it can be observed that maximum concentration (Cmax) of Doxylamine test product i.e. tablet-in-tablet formulation of present study is higher than the marketed sample. Therefore, the present invention has superior effects as compared to marketed sample.

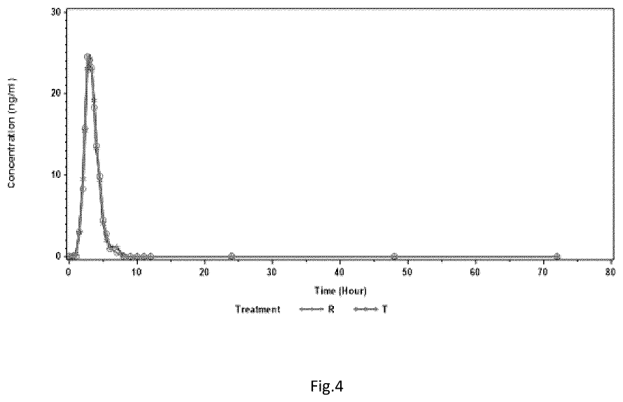

The FIG. 4 represent the linear Plot of Mean Plasma Concentrations of Pyridoxine vs. Time for Test Product (T) and Reference Product (R). From the graph it can be observed that maximum concentration (Cmax) of Pyridoxine test product i.e. tablet-in-tablet formulation of present study is higher than the marketed sample.

We claims:

1. A tablet-in-tablet dosage form comprising:
a. an inner core tablet comprising from about 10 to 20 wt. % Doxylamine and/or a pharmaceutically acceptable salt thereof and from about 10 to 20 wt. % Pyridoxine and/or a pharmaceutically acceptable salt thereof based on total weight of inner core tablet;
b. an outer shell comprising from about 1 to 10 wt. % Doxylamine and/or a pharmaceutically acceptable salt thereof and from about 1 to 10 wt. % Pyridoxine and/or a pharmaceutically acceptable salt thereof based on total weight of outer shell
wherein outer shell is compressed over inner core tablet.

2. A tablet-in-tablet as claimed in claim 1 wherein the inner core tablet is coated with enteric coating polymer which provides delayed release of the active agents.

3. A tablet-in-tablet as claimed in claim 2 wherein enteric coating polymer is Eudragit L present in an amount ranging from 5-10 wt. % more preferably 5.69 wt. % based on total weight of inner core tablet.

4. A tablet-in-tablet as claimed in claim 1-3 the inner core tablet further comprises one or more acceptable excipients selected from binder, diluents, disintegrants, glidant, wetting agent, lubricants.

5. A tablet-in-tablet as claimed in previous claims comprises an inner core having:
a. From about 15.5 wt. % Doxylamine succinate and 16.92 wt. % Pyridoxine hydrochloride based on total weight of inner core tablet;
b. From about 0.75 wt. % Polyvinylpyrrolidone (PVPPK-30) based on total weight of inner core tablet as binder;
c. From about 5-40 wt. % microcrystalline cellulose, starch and Dibasic calcium phosphate based on total weight of inner core tablet as diluents;
d. From about 0.5-5 wt. % of croscarmellose sodium, crosspovidone and sodium starch glycollate based on total weight of inner core tablet as disintegrant;
e. From about 1.89 wt. % of colloidal silicon dioxide based on total weight of inner core tablet as glidant;
f. From about 0.90 wt. % of Sodium Lauryl sulfate based on total weight of inner core tablet as wetting agent;
g. From about 1.13 wt. % of Magnesium stearate based on total weight of inner core tablet as lubricant;
h. From about 5.69 wt. % of Eudragit L as enteric coated polymer which provides delayed release of active agents.

6. A process of preparation of inner core tablet as claimed in claim 1 wherein the process comprises following process steps:
a. Dry mixing of active agents with diluents and disintegrant.
b. Wet granulating the premix with binder solution to form granules
c. Drying the wet granules and lubricating the dried granules
d. Compressing the lubricating granule into a tablet
e. Coating the tablet with one more seal coating
f. Enteric coating the seal granules with delayed release polymer

7. A tablet-in-tablet as claimed in previous claims comprises an outer shell having:
a. From about 1-10 wt. % Doxylamine succinate and Pyridoxine hydrochloride based on total weight of outer shell and;
b. From about 10-50 wt. % microcrystalline cellulose, starch and Dibasic calcium phosphate based on total weight of outer shell as diluents and;
c. From about 0.5-5 wt. % croscarmellose sodium based on total weight of outer shell as disintegrate and;
d. From about 0.5-5 wt. % Polyvinylpyrrolidone (PVPPK-30) based on total weight of outer shell as binder and;
e. From about 0.05-2 wt. % magnesium stearate and talcum based on total weight of outer shell as lubricant and;
f. From about 0.5-3 wt. % colloidal silicon dioxide based on total weight of outer shell as glidant

8. A tablet-in-tablet dosage form as claimed in claim 1, wherein outer shell provides immediate release of the active agents.

9. A process of preparation of outer shell tablet as claimed in claim 1, wherein the process comprises following process steps:
a) Dry mixing diluent and disintegrants with active agents
b) Wet granulating the dry mix with binder solution to form wet granules
c) Drying the wet granules and lubricated the dried granules with the lubricants
d) Compressing the dried granules with inner core tablet to form tablet-in-tablet formulation
e) coating the outer shell of tablet-in-tablet with seal coating
wherein outer shell may be further coated with seal coating having plasticizer, film former and solvent and optionally colorant.

10. A tablet-in-tablet dosage form wherein the dosage form as claimed in any of the above claim, wherein the outer shell is multi-layered more preferably bi-layered.

11. A tablet-in-tablet dosage form as claimed in claim 10, wherein the bi-layered outer shell is composed of an upper layer and a lower layer wherein the amount of doxylamine succinate and pyridoxine hydrochloride is split between the upper layer and the lower layer is in the ratio of 1:1.

12. A tablet-in-tablet dosage form as claimed in any of the above claims, wherein the dosage form is used for the treatment of nausea and vomiting in pregnancy.
